(19) United States
(12) Patent Application Publication
(10) Pub. No.: US 2015/0031104 A1
(43) Pub. Date: Jan. 29, 2015

(54) System For Generating Biogas And Method For Operating Such A System

(71) Applicant: Karlgünter Eggersmann, Marienfeld (DE)

(72) Inventor: Karlgünter Eggersmann, Marienfeld (DE)

(21) Appl. No.: 14/128,215

(22) PCT Filed: Apr. 7, 2012
(86) PCT No.: PCT/EP2012/001548

§ 371 Date: Sep. 12, 2014

(30) Foreign Application Priority Data
Jun. 24, 2011 (EP) 11005135.6

(51) Int. Cl. C12M 1/107; C12M 1/00; C12P 5/02

(52) U.S. Cl. CPC C12M 21/04; C12P 5/023; C12M 29/26; C12M 23/36; C12M 29/02; C12M 29/18
USPC 435/167; 435/300.1; 435/297.1

(57) ABSTRACT

A system for generating biogas comprises at least one fermenter (1) for fermenting solid biomass (13). The fermenter (1) comprises a device for charging the biomass with a liquid medium, a gas outlet (2) for removing the biogas from the fermenter (1) and a gas inlet (3) for supplying gases to the fermenter (1). The gas inlet (3) is connectable to a biogas source, preferably to the gas outlet (2), a biogas line and/or a biogas reservoir.

### TECHNICAL FIELD

The invention concerns a layout for generating biogas according to the preamble of claim 1, as well as a method for operating a layout for generating biogas according to the preamble of claim 9.

### PRIOR ART

By biogas is meant a methane-rich gas which is produced in the anaerobic digesting of biomass. The anaerobic digesting in this context is also known as fermenting or fermentation. The layouts that are known in the prior art for the fermenting of solid biomass essentially have a generally gas-tight fermenter to receive the solid biomass. This can be a garage or tunnel shaped building in which the biomass is placed, for example, in the form of a pile.

Fermenters of the prior art furthermore usually have a device for subjecting the biomass to a liquid medium. The liquid medium is usually a so-called percolate, which passes through the biomass that is normally present in the form of a loose pile or stack and thus has a certain porosity. The percolate also serves in particular as a substrate for the anaerobic biology which is responsible for the fermentation process and usually makes possible the fermenting of the solid biomass that is wetted and thus moistened by the percolate. Since the biomass is not mashed as in a classical wet fermentation, but rather largely retains its pile structure at least in the beginning, one also calls such processes a dry fermentation, even though strictly speaking the fermentation itself takes place in a moist medium, namely, one wetted by the percolate.

The fermenters of the kind in question generally have a gas outlet to take away the biogas from the fermenter and a gas inlet for supplying gases to the fermenter. The gas outlet serves at least primarily to take away the biogas from the fermenter, while the gas inlet is generally used to flush the fermenter, for example, to displace the oxygen from the fermenter with a gas containing carbon dioxide so as to lessen the explosion danger in the transition from aerobic to anaerobic phase, by taking away explosive gas mixtures or gases that can result in the formation of such mixtures.

Such flushing processes, which need to be carried out especially when the fermenter is being run up and run down, and during the associated switching between aerobic and anaerobic processes, bring with them the problem that a large amount of gas with a no longer technically usable composition is formed during the flushing, and it must generally be taken to special torches or filter systems for disposal. Especially due to the porosity of the solid in the fermenter, a substantial amount of biogas is lost in this way, making the process inefficient.

### THE INVENTION

The problem of the invention is to indicate a layout for the generating of biogas and a method for operating a layout for generating of biogas that enable a more efficient generation of biogas, especially in regard to the avoiding of gas losses by flushing processes and a higher possible content of dry substrate.

The problem is solved by a layout with the features of claim 1 and a method with the features of claim 9. Advantageous modifications will be found in the subclaims.

According to the invention, the problem is solved in that biogas is introduced into the fermenter. For this purpose, according to the invention the gas inlet of the fermenter can be connected to a biogas source, preferably the gas outlet of the same and/or another fermenter, a biogas pipeline, or a biogas reservoir. This is generally done by pipelines and shutoff fittings with which the pipeline connections needed for the particular process stage can be made and broken as necessary. In layouts that have a plurality of fermenters, it is advantageous to make it possible to connect the gas outlets of the various fermenters to a common biogas pipeline. The biogas being introduced into a fermenter can advantageously be taken from the common biogas pipeline, which brings the benefit that the biogas supply can be provided more reliably by the shared use of the biogas pipeline, than is the case when it is removed from a single fermenter. Especially when the fermenters operating on the common biogas pipeline are being operated such that the different process stages of the fermenter operation, especially the run-up and run-down operation, are staggered in time in different fermenters, so that always at least one fermenter is in a desirable biogas-producing phase.

It is advantageous for the fermenter or fermenters to have an additional gas outlet besides the biogas outlet, or for the biogas outlet to have a switching or shutoff branch that leads to a suitable gas disposal arrangement, such as a torch, especially a lean gas torch, or a biofilter or other exhaust gas treatment. If gases arise during run-up or run-down processes or flushing processes that cannot be supplied to the biogas system for utilization, they can be disposed of and/or handled appropriately via the other gas outlets or branches.

Preferably, the layout has a delivery arrangement for delivering the biogas in the direction of the gas inlet. This delivery arrangement can be, for example, a compressor or supercharger, which is able to deliver the biogas in the direction of the fermenter. It is advantageous, on the one hand, that the correct flow directions in the biogas pipeline system can be assured in this way, and on the other hand the biogas can also be delivered to the fermenter against pressure resistance, which is especially advantageous when the biogas is conveyed to the fermenter in such a way that the biogas is forced to flow through the biomass.

As already mentioned above, the biomass pile or stack has an inner porosity or cavities on its inside, in which gas collects; in particular, this may involve air inclusions, which are present in the biomass after the biomass is placed in the fermenter. In order to eliminate these air inclusions as efficiently as possible, it is advisable to convey the biogas into the fermenter in such a way that the biogas is forced to flow through the biomass. A further benefit of this procedure is that drainage channels can be created in the pile in this way. For this purpose, it is especially advantageous to introduce biogas into the fermenter during the fermentation phase, i.e., during the supply of the liquid medium to the fermenter and the subjecting of the solid biomass to the liquid medium. The creating of the drainage channels is advantageous in this case, even if no other gas is displaced by the biogas when the biogas is introduced in this phase of the process. It can be especially advisable in this phase to run the biogas of a single fermenter in a circuit.

The forced flow through the biomass preferably occurs from the bottom of the fermenter in a primarily vertical direction. In this way, especially when the gas inlet is designed as a gas distributing arrangement, especially as a spigot floor, a network of drainage channels can be created in the pile or stack of solid biomass, making possible the most homogeneous possible flow of the liquid medium through the solid biomass. Alternatively, it is also possible to introduce the flushing gas at the side into the pile. Regardless of the direction of flow, the better dewatering of the pile is a further benefit.

The gas inlet is therefore preferably arranged beneath the biomass being introduced into the fermenter, preferably in the floor of the fermenter. This is especially advisable when an arrangement for lateral support of the solid biomass is provided inside the fermenter, preferably permeable to liquids. The providing of such a support is advantageous to making possible a tall stack height and at the same time making possible an outflow of the liquid medium from the stack or pile of biomass at the side. However, this design is prone to creating a dead volume in the lower middle region of the pile, which is not adequately reached by the flow of liquid. This can be effectively prevented by the advantageous placement of the gas inlet beneath the biomass. This also makes possible an advantageous drainage of the liquid medium at the bottom.

Thus, the inflow of gas or biogas and the drainage of the liquid medium occurs through the same gas inlet. This makes it possible for the drainage channels created by the biogas blown in to always lead to the outlet.

Especially advantageously, after the end of the supplying of the liquid medium, the biogas can be channeled into the fermenter to support the dewatering of the biomass. In this way, it is possible to maintain the fermentation process until reaching the desirable content of water or dry substrate for removal of the biomass from the fermenter. Thus, only a minimal short flushing process of the fermenter is needed between the fermentation phase under biogas atmosphere for generating of the biogas and the removal of the fermented biomass, which necessarily involves an aeration of the fermenter.

It is likewise advantageous for the biogas being used for flushing the fermenter during the run-up of the fermenter to be used advantageously for the displacing of gases, especially oxygen and nitrogen, from the fermenter and especially from the porosities and/or cavities of the biomass in the fermenter. In this way, already after a short flushing process one can commence the anaerobic fermentation and generate usable biogas. This avoids the formation of technically unusable gas mixtures during the run-up operation. To accomplish the flushing during run-up and run-down processes it is especially advantageous for the layout to have a plurality of fermenters and for the gas inlet and gas outlet of different fermenters to be connectible to each other. This can be done, for example, by a switching to the common biogas pipeline. Preferably, a biogas reservoir can also be provided, which can be connected preferably to the gas outlet and/or gas inlet of preferably all available fermenters and which serves to assure the availability of the biogas.

### BRIEF DESCRIPTION OF THE DRAWINGS

The invention will be explained more closely below by means of FIGS. 1 to 4:

FIG. 1 shows a flow chart of a sample embodiment of the invention,

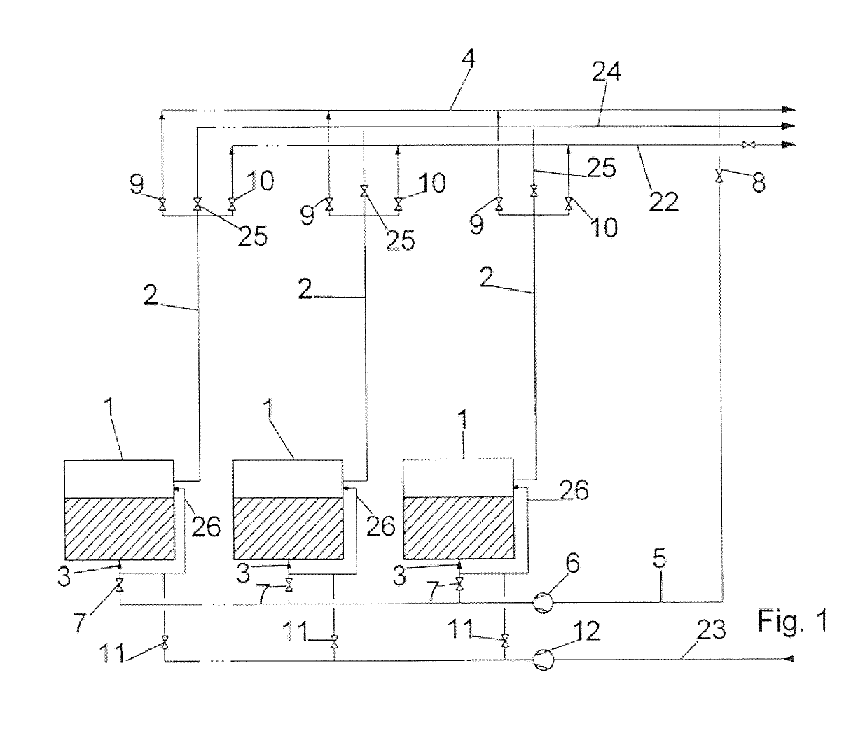

FIG. 2 shows schematically the flow pathways of the percolate and the creation of a dead volume without the biogas supply according to the invention,

FIG. 3 shows schematically the flow pathways of supplied biogas through the biomass in a sample embodiment of a fermenter according to the invention,

FIG. 4 shows schematically examples of flow pathways of the liquid medium through the biomass after creating additional drainage channels with an advantageous flushing process according to the invention.

### BEST WAY OF IMPLEMENTING THE INVENTION

The sample layout has a plurality of fermenters 1, whose gas outlets 2 can be connected via the shutoff fittings 9 to the common biogas pipeline 4. Furthermore, it is possible to connect the gas outlets 2 to the additional exhaust gas pipeline 22 by means of the shutoff fittings 10. The sample layout, furthermore, has an additional protection gas pipeline 24, which can be connected to the fermenters 1 by means of the shutoff fittings 25.

The gas inlets 3 of the individual fermenters can be connected to the common biogas pipeline 4 via the shutoff fitting 8 and the pipeline 5. The gas inlets 3 can be connected individually by their respective shutoff fittings 7 to the pipeline 5 or be disconnected from it. In the pipeline 5, a delivery arrangement 6 is provided, for example, a compressor, in order to ensure the delivery of the biogas to the gas inlets. In addition, the layout according to the invention has another flushing gas supply line 23, with which air, for example, can be delivered to the gas inlets in the floors of the fermenters 1 and/or other gas inlets 26 in the region of the head space of the fermenters 1. For this purpose, an additional delivery arrangement 12 is provided. The individual gas inlets 3 can be connected to this pipeline by the shutoff fittings 11.

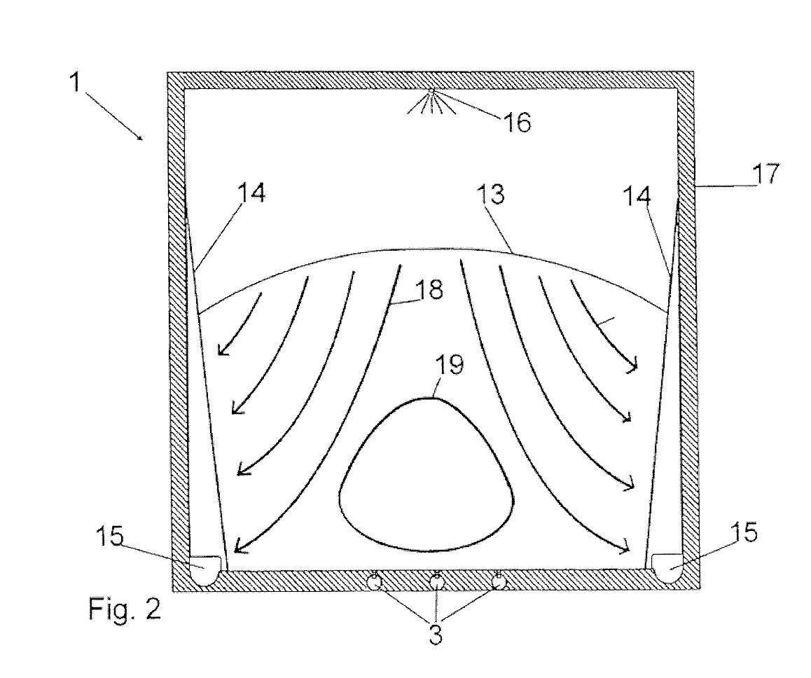

The sample fermenters 1 according to the invention have gas inlets 3 in their floor, designed as a spigot floor. Furthermore, inside the gas-tight shell 17 of the fermenter is arranged a device 16 for subjecting the biomass 13 to a liquid medium. The biomass 13 in the case of the sample layout is supported at the sides by an arrangement 14 that is permeable to the liquid medium. The tank in the case of the sample layout furthermore has collecting channels 15 to take away the liquid medium. Without the use of the flushing according to the invention for the creating of additional drainage channels in the pile of biomass 13, the liquid medium in penetrating the pile will take the preferred flow pathways 18 indicated in FIG. 2. This produces a dead volume 19 in the lower middle region of the pile of biomass 13, which is not adequately reached by the liquid medium flow.

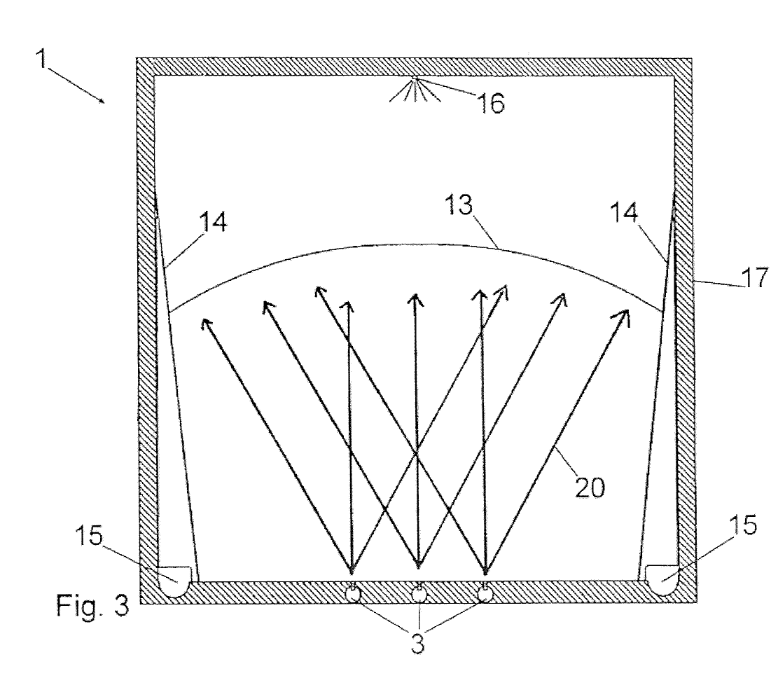

If biogas is introduced into the fermenter 1 through the gas inlet 3, the pile of biomass 13 is subjected to a forced flow of the introduced biogas, and a primarily vertical flow through the biomass 13 arises thanks to the arrangement of the gas inlet 3 in the floor of the fermenter. The prevailing flow pathways and directions 20 in this case are shown schematically in FIG. 3.

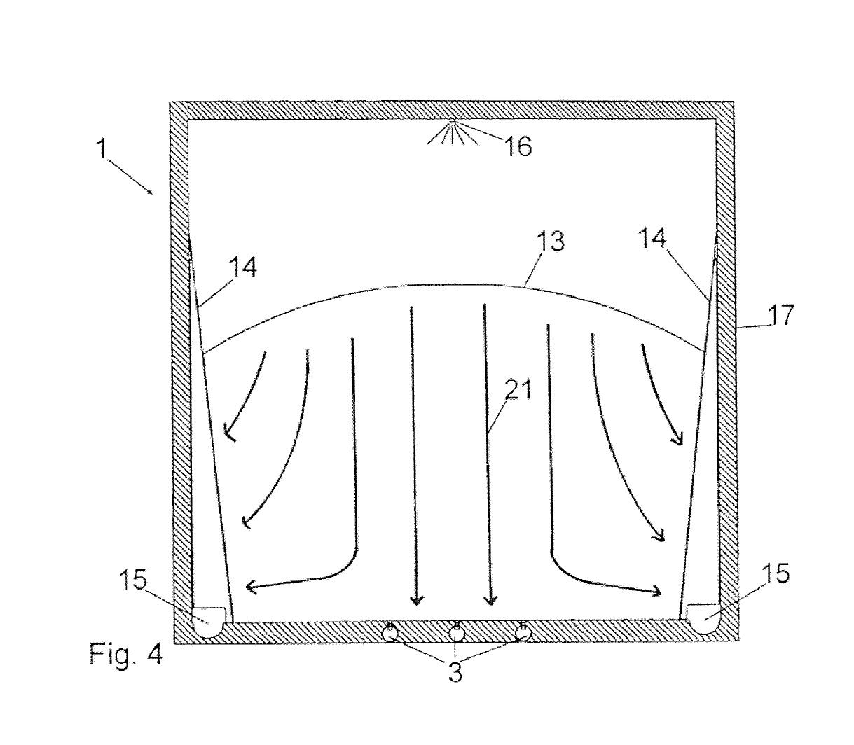

Thanks to the additional drainage channels created in this way in the pile of biomass 13, a more homogeneous flow through the biomass 13 and a suppression of the formation of dead volumes are accomplished, as is shown by the schematically illustrated flow pathways 21 in FIG. 4. The liquid medium, after passing through the biomass 13, is taken away via the connecting channels 15 and/or the gas inlets 3.

1. Layout for the generating of biogas, which has at least one fermenter (1) for the fermenting of solid biomass (13), wherein the fermenter (1) has a device for subjecting the biomass (13) to a liquid medium, a gas outlet (2) for taking away the biogas from the fermenter (1) and a gas inlet (3) for supplying gases to the fermenter (1), wherein the gas inlet (3) can be connected to a biogas source, preferably to the gas outlet (2), a biogas pipeline and/or a biogas reservoir, characterized in that the gas inlet (3) is designed such that the liquid medium after passing through the biomass is drained by means of it, and biogas is introduced into the fermenter during the supplying of the liquid medium to the fermenter (1).

2. Layout according to claim 1, characterized in that the gas inlet (3) is configured as a gas distributing arrangement, especially a spigot floor.

3. Layout according to claim 1, characterized in that
the layout has a delivery arrangement (6) for delivering the biogas in the direction of the gas inlet (3).

4. Layout according to claim 1, characterized in that the layout has a biogas reservoir, which can be connected preferably to the gas outlet (2) and/or to the gas inlet (3)

5. Layout according to claim 1, characterized in that an arrangement (14) for lateral supporting of the biomass (13) is provided inside the fermenter, being preferably permeable to liquids.

6. Layout according to claim 1, characterized in that the gas inlet (3) is arranged underneath the biomass being introduced into the fermenter (1), preferably in the floor.

7. Layout according to claim 1, characterized in that the layout has a plurality of fermenters (1) and gas inlet (3) and gas outlet (2) of different fermenters (1) can be connected to each other.

8. Layout according to claim 7, characterized in that a plurality of gas outlets (2) of different fermenters (1) can be connected to a common biogas pipeline (4).

9. Layout according to claim 1, characterized in that the gas inlet is also configured for taking away the liquid medium from the fermenter (1).

10. Method for operating a layout for generating of biogas according to claim 1, wherein solid biomass (13) is introduced into a fermenter (1), subjected to a liquid medium and fermented anaerobically to generate biogas, wherein biogas is introduced into the fermenter (1), characterized in that
the biogas is conveyed into the fermenter (1) during the supply of the liquid medium through gas inlets (3) so that drainage channels for the liquid medium are created, whereupon the liquid medium after passing through the biomass (13) is taken away via the gas inlets (3).

11. Method according to claim 10, characterized in that the biogas is channeled into the fermenter (1) to support the drying of the biomass after the end of the supplying of liquid medium.

12. Method according to claim 10, characterized in that
the biogas is channeled into the fermenter (1) during the run-up of the fermenter (1) to displace gases, especially oxygen and nitrogen, from the fermenter (1), especially from porosities and/or cavities of the biomass (13).

13. Method according to claim 10, characterized in that the biogas is channeled into the fermenter such that a forced flow of the biogas through the biomass (13) is accomplished.

14. Method according to claim 13, characterized in that the forced flow through the biomass (13) starts from the bottom of the fermenter (1) and occurs in primarily vertical direction.

15. Method according to claim 10, characterized in that the biogas is taken from a fermenter (1) of a common biogas pipeline (4) of several fermenters (1) or a biogas reservoir other than the one into which it is being introduced.

16. Method according to claim 10, characterized in that the taking away of the liquid medium from the fermenter (1) occurs through the gas inlet (3).
